(19) United States
(12) Patent Application Publication
(10) Pub. No.: US 2005/0215957 A1
(43) Pub. Date: Sep. 29, 2005

(54) Calibrated pushrod for injection volume control in prefilled syringes

(75) Inventor: Michael R. Hynes, Chesterfield, MO (US)

Correspondence Address: WOOD, HERRON & EVANS, LLP, 2700 CAREW TOWER, 441 VINE STREET, CINCINNATI, OH, 45202, US

(73) Assignee: Mallinckrodt Inc., St. Louise, MO (US)

(21) Appl. No.: 10/811,258

(22) Filed: Mar. 26, 2004

(51) Int. Cl. A61M 5/315

(52) U.S. Cl. 604/218

(57) ABSTRACT

A pushrod for use with a prefilled syringe containing a known amount of a medical fluid and having a volume, including a shaft having a scale corresponding to the cross-sectional area of the prefilled syringe, a stop configured for location along the shaft, the stop located using the scale in correspondence with a prescribed dosage injected from the prefilled syringe.

### FIELD OF THE INVENTION

The present invention relates generally to syringes for injecting fluids into a subject and more particularly to injecting only some portion of the contents of a prefilled syringe.

### BACKGROUND OF THE INVENTION

In many medical environments, a medical fluid is injected into a subject or patient during testing, diagnosis or treatment. One example is the injection of contrast media into a patient to improve CT, Angiographic, Magnetic Resonance, nuclear medicine or Ultrasound imaging, using a prefilled hand-held syringe.

Prefilled syringes suitable for these and similar applications are available in a variety of sizes, such as, for example, 10, 15, 20, 30, and 50 milliliters (mL). Despite the availability of numerous sizes, dosages prescribed for patients often fall intermediate two sizes.

When a prescribed dosage falls between two sizes, a clinician generally selects the syringe sized just larger than the prescribed dosage, thereby minimizing fluid waste. The clinician then subtracts the prescribed dosage from the selected syringe size to arrive at the amount of fluid that should remain in the syringe once the dosage has been administered. The clinician must then observe markings found on the side of the syringe, injecting until the calculated amount of fluid that should remain in the syringe once the prescribed dosage has been administered is reached.

For example, suppose a scenario wherein the prescribed dosage is 18 mL. As 18 mL falls between 15 and 20 mL, a clinician would likely select a 20 mL prefilled syringe. The clinician then subtracts 18 mL from 20 mL, arriving at 2 mL, or the amount of fluid that should remain in the syringe once the prescribed dosage has been administered. The clinician then observes the markings on the side of syringe, injecting until 2 mL of fluid remains in the syringe.

With a dosage of 18 mL the mathematics involved are not particularly difficult or troublesome. However, clinicians are commonly under a tremendous amount of pressure in the workplace, often facing life and death situations with patients under their care. Physicians, physician assistants, residents, nurses, etc. also commonly work extended shifts, many times lacking adequate rest. Moreover, clinicians often administer multiple injections in a given shift. In such circumstances, clinicians conducting even the most elementary calculations are prone to errors having potentially grave results for patients under their care.

Now suppose another scenario wherein the prescribed dosage is 9.6 mL and the only size prefilled syringe available is 15 mL. In such a scenario, the clinician must subtract 9.6 mL from 15 mL, arriving at 5.4 mL. Clearly, such a computation whether done mentally, in writing, or with a calculator, is more difficult, time consuming, and/or prone to error than the calculation in the previous scenario. Add in the attendant workplace pressures, and a lack of rest, and, clearly, there is the potential for injecting a patient with an incorrect dosage due to clinician error.

Accordingly, a need exists to simplify the injection procedure used with prefilled syringes so that a clinician no longer needs to perform a calculation to arrive at the amount of fluid that should remain in the syringe once the dosage has been administered or observe the markings on the side of syringe when injecting a medical fluid into a patient.

Another environment of relevance is in nuclear medicine and Positron Emission Tomography (PET) procedures. Due to the radioactive nature of the radiopharmaceutical agents used for those procedures, great care is taken to reduce the clinician's exposure to the radiation. When injecting radiopharmaceuticals, the clinician typically covers the syringe with a radiation blocking safety shield. The safety shield is typically made from a heavy metal such as lead or tungsten and many designs do not have provisions to see the volume markings on the syringe barrel. This increases the challenge of providing a set volume injection.

Another issue that arises, is the responsibility of the clinician to observe any physiological changes in the patient during the injection to identify possible adverse reaction. It can be difficult for the clinician to direct their attention to the patient when focusing on delivered volume.

### SUMMARY OF THE INVENTION

The needs identified above and other problems of conventional injection procedures used with prefilled syringes are addressed by embodiments of the present invention which simplifies the injection procedure with prefilled syringes so that a clinician may simply couple a calibrated pushrod to a prefilled syringe, set a stop on the pushrod corresponding to a prescribed dosage, and administer the dosage through injection.

These and other features, aspects, objects, and advantages of the present invention will be made apparent from the accompanying drawings and the description thereof.

### BRIEF DESCRIPTION OF THE DRAWINGS

FIG. 1 illustrates a side view of a hand-held syringe assembly in accordance with principles of the present invention. The assembly includes a prefilled syringe, shown partly broken away, and a calibrated pushrod for use therewith.

FIG. 2 is a partial side view of the hand-held syringe assembly of FIG. 1, but with the calibrated pushrod positioned after injecting a prescribed dosage of 3 milliliters (mL).

### DETAILED DESCRIPTION OF THE INVENTION

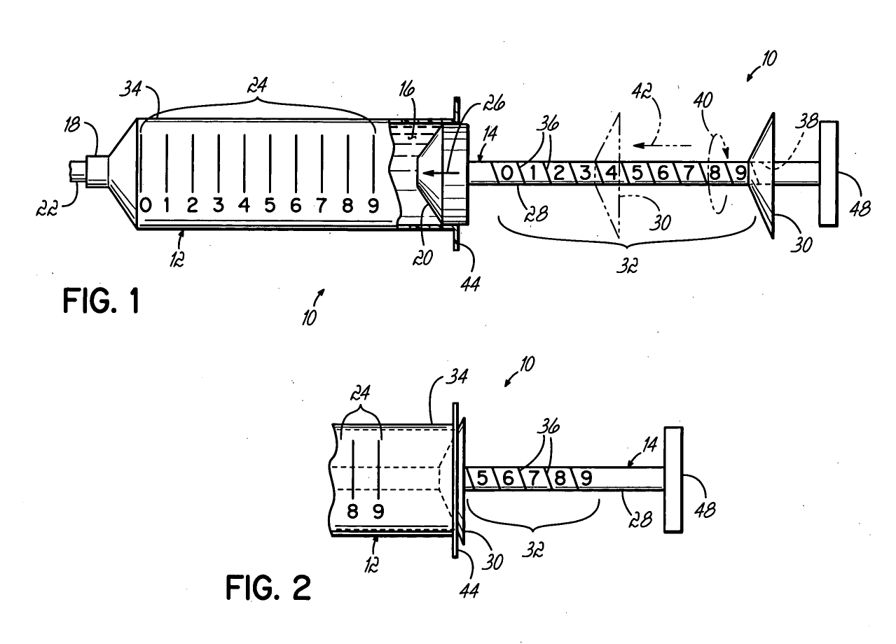

With reference to FIG. 1, there is shown one embodiment of a hand-held syringe assembly 10 in accordance with the principles of the present invention. Assembly 10 comprises a prefilled syringe 12 and a calibrated pushrod 14 configured for use with the prefilled syringe.

Prefilled syringes are currently available containing a variety of medical fluids, and are commonly used in procedures involving patient test, diagnosis or treatment. Further, prefilled syringes suitable for these and similar applications are available in a variety of sizes, such as, for example, 10, 15, 20, 30, and 50 milliliters (mL), the sizes relating somewhat to typical and/or average dosages. For example, prefilled syringe 12 is a 10 mL syringe containing 10 mL of a contrast media 16, a portion of which may be injected into a patient to improve CT, Angiographic, Nuclear Medicine and PET, Magnetic Resonance or Ultrasound imaging in accordance with a prescribed dosage.

Prefilled syringe 12 is generally cylindrically shaped, comprising a barrel 34 with a flange 44 formed at one end. Formed at the other end of the barrel 34 is a nozzle 18. Inserted axially at the flanged end of the barrel 34, as indicated by arrow 26, and viewable due to a portion of the barrel being partly broken away, is a plunger 20. In transit, a cap or seal (not shown) may be placed over the end of the syringe into which the plunger is inserted. Likewise, the tip 22 of nozzle 18 might also be capped or sealed (also not shown). Such caps or seals generally prevent unwanted dispensing of the fluid contents of prefilled syringes in transit or storage.

Prefilled syringe 12 further includes current volume markers 24 on barrel 34, indicating the volume of contrast media 16 remaining in syringe 12. Markers 24 are based on the cross-sectional of the barrel 34 and axial displacements of plunger 20 along the axis of the barrel 34, likewise, indicated by arrow 26. As illustrated, markers 24 read from 0 to 9 mL.

In administering a prescribed dosage one end of an extension tube (not shown) is slid over tip 22 of nozzle 18 on prefilled syringe 12, the other end of the extension tube leading to an injection site on a patient, such as though an intravenous tube. The tips of nozzles of prefilled syringes may also include threads onto which needles may be threaded, thereby allowing needle-stick injection.

Calibrated pushrod 14 includes a shaft 28 and a stop 30. Embossed on or formed or molded into shaft 30 is a scale 32, a portion of the molding, for example, providing threads 36. Stop 30 likewise includes threads 36, shown in hidden lines, located on the interior bore 38 of the stop, and corresponding to the threads molded into the shaft. Thus, stop 30 may be threaded onto shaft 28, the location of the stop along the shaft being determined by how far the stop is threaded onto the shaft.

Scale 32 like markers 24 corresponds to the cross-sectional area of prefilled syringe 12, and may be thought of by those of skill in the art, as transposing current volume markers 24 onto shaft 28. Thus, scale 32 like markers 24, reads from 0 to 9 mL, and pushrod 14 is calibrated to the size of prefilled syringe 12, and calibrated pushrod 14 may only be properly used with like-sized syringes, i.e., 10 mL.

For example, and in use, shaft 28 of calibrated pushrod 14 is coupled to plunger 20 of prefilled syringe 12. Assuming a prescribed dosage of 3 mL, for example, stop 30 is rotated, as indicated by arrow 40, such that stop 30 is moved along shaft 28, as indicated by arrow 42, locating the stop, as shown in phantom, at the 3 mL mark on scale 32.

Turning now to FIG. 2, a side view of hand-held syringe assembly 10 is shown with stop 30 located at the 3 mL mark and calibrated pushrod 14 positioned as it would be after a clinician had injected the prescribed dosage of 3 mL. As illustrated, pushrod 14 and plunger 20 are prevented from traveling any further and stop when stop 30 contacts flange 44 of barrel 34, thereby controlling the injection volume, and allowing only 3 mL of contrast media 16 to be injected in accordance with the prescribed dosage.

Referring also to FIG. 1, such an injection may be performed by a clinician by placing prefilled syringe 12 between index and middle fingers of one hand, and placing the thumb on thumb rest 48 of shaft 28. The clinician then squeezes the fingers and thumb together causing calibrated pushrod 14 to slide plunger 20 within barrel 34 of prefilled syringe 12, discharging contrast media 16 out tip 22 of nozzle 18, thereby performing the injection.

Thus, the present invention simplifies the injection procedure commonly used with prefilled syringes so that a clinician no longer needs to perform a calculation to arrive at the amount of fluid that should remain in the syringe once the dosage has been administered or observe the markings on the side of syringe when injecting a medical fluid into a patient. For example, a clinician merely couples calibrated pushrod 14 to prefilled syringe 12, sets stop 30 on pushrod 14 to the prescribed dosage of 3 mL, and administers the dosage through injection.

While the present invention has been illustrated by description of a particular embodiment and while this embodiment has been described in considerable detail, it is not the intention of the applicant to restrict or in any way limit the scope of the claims to such detail. Additional advantages and modifications will readily appear to those skilled in the art. For example, prefilled syringes are available in many different sizes and pushrods may be calibrated to any of these sizes. Moreover, present invention was described in the context of medical applications; however, there may be other applications in which a user wishes to use prefilled syringes to inject a specific amount of fluid. The invention in its broader aspect is, therefore, not limited to the specific details, representative system, apparatus, and method, and illustrative example shown and described. Accordingly, departures maybe made from such details without departing from the spirit or scope of the applicant's general inventive concept.

1. A pushrod for use with a prefilled syringe containing a known amount of a medical fluid and having a cross-sectional area, comprising:
a shaft including a scale corresponding to the volume of the prefilled syringe; and,
a stop configured for location along the shaft;
the stop located using the scale in correspondence with a prescribed dosage injected from the prefilled syringe.

2. The pushrod of claim 1, the shaft and the stop including corresponding threads, the threads used for locating the stop along the shaft.

3. The pushrod of claim 1, the shaft including a thumb rest.

4. The pushrod of claim 1, the shaft made of a molded material.

5. The push rod of claim 1, the prefilled syringe including a barrel with a flange, the stop abutting the flange when the prescribed dosage has been injected.

6. A hand-held syringe assembly comprising:
a prefilled syringe containing a known amount of a medical fluid and having a cross-sectional area; and,
a pushrod configured for use with the prefilled syringe and including:
a shaft having a scale corresponding to the volume of the prefilled syringe; and,
a stop configured for location along the shaft;
the stop located using the scale in correspondence with a prescribed dosage injected from the prefilled syringe.

7. The assembly of claim 6, the shaft and the stop including corresponding threads, the threads used for locating the stop along the shaft.

8. The assembly of claim 6, the shaft including a thumb rest.

9. The assembly of claim 6, the shaft made of a molded material.

10. The assembly of claim 6, the prefilled syringe including a barrel with a flange, the stop abutting the flange when the prescribed dosage has been injected.

11. A method of injecting a medical fluid from a prefilled syringe containing a known amount and having a cross-sectional area, comprising:
coupling a calibrated pushrod to the prefilled syringe;
setting a stop on the pushrod to a prescribed dosage; and,
administering the dosage through injection.
